(19) United States
(12) Patent Application Publication
(10) Pub. No.: US 2026/0334880 A1
(43) Pub. Date: Oct. 29, 2026

(54) SENSOR MOUNT FOR INTRAVASCULAR DEVICE WITH INTEGRATED CONDUCTIVE PADS FOR ELECTRICAL CONNECTION TO SENSOR

(71) Applicant: KONINKLIJKE PHILIPS N.V., EINDHOVEN (NL)

(72) Inventors: Bernal CORDERO, ALAJUELA (CR); Diego Alonso BONILLA MONTERO, ALAJUELA (CR)

(21) Appl. No.: 19/485,357

(22) PCT Filed: May 24, 2024
(86) PCT No.: PCT/EP2024/064449

§ 371 Date: Nov. 18, 2025

Related U.S. Application Data
(60) Provisional application No. 63/472,916, filed on Jun. 14, 2023.

(51) Int. Cl. A61B 5/00; A61B 5/0215; A61M 25/09

(52) U.S. Cl. CPC A61B 5/6851; A61B 5/02158; A61B 2560/0443; A61M 2025/09175

(57) ABSTRACT

An intraluminal device is provided. The intraluminal device includes a flexible elongate member configured to be positioned within a body lumen of a patient and a sensor mount positioned at a distal portion of the flexible elongate member. The sensor mount includes a first plurality of conductive pads. The sensor mount additionally includes a sensor assembly. The sensor assembly includes a sensor and a second plurality of conductive pads. The second plurality of conductive pads is electrically and mechanically coupled with the first plurality of conductive pads.

### TECHNICAL FIELD

The present disclosure relates generally to intravascular measurement devices (e.g., intravascular catheter or guidewires). In particular, a sensor mount includes multiple conductive pads that allow electrical connection to a sensor mounted on the sensor mount (e.g., an intravascular pressure sensor mounted on the sensor mount).

### BACKGROUND

Intravascular measurement devices are commonly used in medical diagnosis to provide insights to a physician regarding the health of a patient. For example, intravascular measurement devices may acquire pressure, flow, or image data from within a lumen of a patient, such as a blood vessel. This data may be used by the physician to diagnose various conditions as well as formulate treatment plans. By nature, intravascular measurement devices are small and flexible and are designed to be positioned and moved through a lumen of a patient. Assembly of such devices can, therefore, be quite complex.

In particular, current methods of assembly of intravascular measurement devices, such as an intravascular pressure measurement device designed to measure blood pressure within a blood vessel, include manual processes. These include the use of tweezers to mount to a sensor mount. Typically, a set of conductive wires are threaded through an opening in a housing and the sensor is placed within the opening of the housing. The wires must then be electrically and mechanically coupled to the sensor.

This manual process often results in damage to the wires and the sensor. Because the components are so small and delicate, it can be difficult to handle them with tweezers without causing damage. The damage can occur during any step of the assembly process, from mounting the wires to the sensor to fastening the sensor to the mount. In addition, in many cases, it may not be immediately obvious that damage has occurred. The damage may not be detected until the device is fully assembled and tested, which can be a time-consuming and costly process. If the damage is detected at this stage, it may be necessary to disassemble the device and start the assembly process over again, which can further increase costs and delay time-to-market.

### SUMMARY

The present disclosure introduces a sensor mount designed for automated assembly of an intravascular measurement device (e.g., intravascular catheter or guidewire). An intravascular sensor is mounted on the sensor mount (e.g., an intravascular pressure sensor mounted on the pressure sensor mount). The sensor mount provides electrical signal connection between the sensor at the distal portion of the intravascular device and conductors that extend from the distal portion to the proximal portion of the intravascular guidewire. In that regard, the sensor mount is both a physical mount and an electrical interposer for the sensor. The sensor mount helps reduce manual assembly, decreasing risk of damage and increasing assembly speed and efficiency. The mount includes two proximal conductive pads and three distal conductive pads, and a region for bonding the sensor assembly via a machine. Two of the distal pads carry positive and negative signals, which are isolated but in electrical communication with the proximal pads. These pads are connected to electrical conductors extending to a control system outside the patient's body. The third distal pad corresponds to a ground signal, which is in electrical and mechanical communication with the sensor mount and a core wire. The device's components are structured to allow automated assembly, enhancing reliability, speed, and efficiency.

In an exemplary aspect, an intraluminal device is provided. The intraluminal device includes a flexible elongate member configured to be positioned within a body lumen of a patient; a sensor mount positioned at a distal portion of the flexible elongate member, wherein the sensor mount comprises a first plurality of conductive pads; and a sensor assembly positioned on the sensor mount, wherein the sensor assembly comprises comprising: a sensor configured to obtain intraluminal medical data associated with the body lumen; and a second plurality of conductive pads electrically coupled to the first plurality of conductive pads.

In some aspects, a number of the second plurality of conductive pads is equal to a number of the first plurality of conductive pads. In some aspects, the sensor mount further comprises a third plurality of conductive pads. In some aspects, the third plurality of conductive pads includes a first conductive pad, a second conductive pad, and a third conductive pad. In some aspects, a portion of one of the conductive pads of the first plurality of conductive pads forms a layer of the first conductive pad of the third plurality of conductive pads. In some aspects, a portion of one of the conductive pads of the first plurality of conductive pads forms a layer of the third conductive pad of the third plurality of conductive pads. In some aspects, the second conductive pad of the third plurality of conductive pads is electrically coupled to ground. In some aspects, the first plurality of conductive pads are positioned within a cavity of the sensor mount. In some aspects, a dielectric filler is positioned within the cavity of the sensor mount. In some aspects, the dielectric filler electrically insulates the first plurality of conductive pads from the sensor mount. In some aspects, the first plurality of conductive pads includes three conductive pads. In some aspects, two of the first plurality of conductive pads are electrically coupled to a corresponding cable extending along the flexible elongate member. In some aspects, the sensor assembly is configured to be mechanically coupled to the sensor mount via an adhesive. In some aspects, the sensor assembly is configured to be mechanically coupled to the sensor mount automatically by a machine. In some aspects, the wire bonds are positioned between the first plurality of conductive pads and the second plurality of conductive pads by a machine. In some aspects, the sensor mount is positioned within a housing. In some aspects, the housing includes an opening with a proximal and distal end, the proximal end positioned adjacent to the first plurality of conductive pads of the sensor mount. In some aspects, a width of the proximal end of the opening is wider than greater than a width of a center portion of the opening.

In an exemplary aspect, an apparatus is provided. The apparatus includes an intravascular guidewire configured to be positioned within a blood vessel and comprising: a sensor mount comprising: a first plurality of conductive pads; a second plurality of conductive pads positioned distal to the first plurality of conductive pads, an intravascular pressure sensor disposed on the sensor mount and comprising a third plurality of conductive pads electrically coupled to the second plurality of conductive pads, wherein the first plurality of conductive pads is electrically coupled to a corresponding plurality of conductors extending along a length of the intravascular guidewire.

In some aspects, a number of the second plurality of conductive pads is greater than a number of the first plurality of conductive pads.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic view of an intravascular sensing system that includes an intravascular device, according to aspects of the present disclosure.

FIG. 2 is a schematic diagram of a processor circuit, according to aspects of the present disclosure.

FIG. 3 is a perspective view of a sensor mount of an intravascular device, according to aspects of the present disclosure.

FIG. 4A is a diagrammatic top view of a sensor assembly coupled to a sensor mount of an intravascular device, according to aspects of the present disclosure.

FIG. 4B is an enlarged diagrammatic top view of a sensor mount of an intravascular device, according to aspects of the present disclosure.

FIG. 5 is a diagrammatic view of a signal path of the intravascular sensing system, according to aspects of the present disclosure.

FIG. 6A is a cutaway perspective view of a sensor mount, according to aspects of the present disclosure.

FIG. 6B is a cross-sectional side view of a sensor mount, according to aspects of the present disclosure.

FIG. 7A is a cutaway perspective view of a sensor mount, according to aspects of the present disclosure.

FIG. 7B is a cross-sectional side view of a sensor mount, according to aspects of the present disclosure.

FIG. 8 is a cross-sectional side view of an intravascular device, according to aspects of the present disclosure.

FIG. 9A is a top view of a housing of an intravascular device, according to aspects of the present disclosure.

FIG. 9B is a top view of a housing of an intravascular device, according to aspects of the present disclosure.

FIG. 9C is a top view of a housing of an intravascular device, according to aspects of the present disclosure.

FIG. 10A is a top view of a sensor assembly and housing of an intravascular device, according to aspects of the present disclosure.

FIG. 10B is a top view of a sensor assembly and housing of an intravascular device, according to aspects of the present disclosure.

FIG. 11 is a flow diagram of a method of assembling an intravascular device, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

Embodiments of the present disclosure are systems, devices, and methods for a sensor mount for automated assembly of an intravascular pressure measurement device. The sensor mount disclosed herein advantageously decreases manual assembly required by allowing machine assembly of the sensor assembly. This advantageously decreases the risk of damage to the part and increases the assembly speed and efficiency of the sensor assembly. Also, the disclosed sensor mount advantageously reduces the number of conductor wires extending along the flexible elongate member.

The sensor mount includes two proximal conductive pads and three distal conductive pads. The sensor mount includes a region where a sensor assembly may be bonded to the sensor mount, for example, via a pick and place machine process. The three distal conductive pads of the mount are positioned adjacent to three corresponding conductive pads of the sensor assembly. A machine may form wire bonds electrically coupling the distal conductive pads of the sensor mount to the three corresponding conductive pads of the sensor assembly. In some aspects, wire bonds may alternatively be referred to as bond wires or conductive traces. Two of the three distal conductive pads may carry a positive and negative signal respectively from the sensor assembly. These two distal conductive pads may be electrically isolated from the sensor mount. These two distal conductive pads are in electrical communication with the two proximal conductive pads. Two electrical conductors may be electrically and mechanically coupled to the proximal conductive pads and may extend along the interior of the intravascular device to a control system outside the patient body. The third distal conductive pad may correspond to a ground signal. The third distal conductive pad may be in electrical and mechanical communication with the sensor mount. The sensor mount may be in electrical and mechanical communication with a core wire which extends along the interior of the interior of the intravascular device and carries the ground signal from the device to the control system. A housing is positioned around the device and includes an opening sized and shaped to accommodate for components of the automated machine used to assembly the device. In this way, the arrangement of the components of the device disclosed advantageously allow the device to be assembled according to an automated process with a machine greatly reducing or completely eliminating manual assembly and increasing reliability, speed, and efficiency.

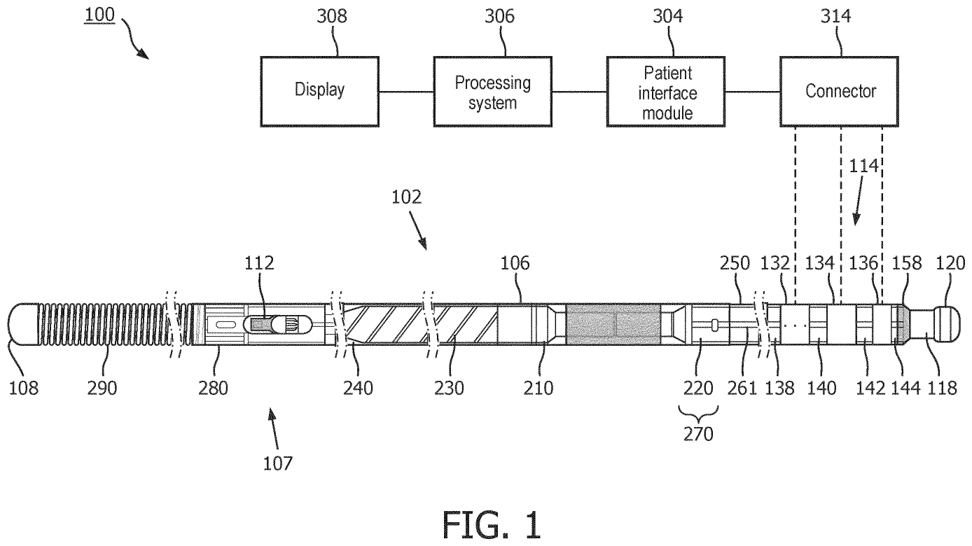

FIG. 1 is a diagrammatic view of an intravascular sensing system 100 that includes an intravascular device 102, according to aspects of the present disclosure. The intravascular device 102 can be an intravascular guidewire sized and shaped for positioning within a vessel of a patient. The intravascular device 102 includes a distal tip 108 and an electronic component 112. For example, the electronic component 112 can be a pressure sensor and/or flow sensor configured to measure a pressure of blood flow within the vessel of the patient, or another type of sensor including but not limited to a temperature or imaging sensor, or combination sensor measuring more than one property. For example, the flow data obtained by a flow sensor can be used to calculate physiological variables such as coronary flow reserve (CFR). The intravascular device 102 includes a flexible elongate member 106. The electronic component 112 is disposed at a distal portion 107 of the flexible elongate member 106. The electronic component 112 can be mounted at the distal portion 107 within a housing 280 in some embodiments. A flexible tip coil 290 extends distally from the housing 280 at the distal portion 107 of the flexible elongate member 106. A connection portion 114 located at a proximal end of the flexible elongate member 106 includes conductive portions 132, 134. In some embodiments, the conductive portions 132, 134 can be conductive ink that is printed and/or deposited around the connection portion 114 of the flexible elongate member 106. In some embodiments, the conductive portions 132, 134 are conductive, metallic bands or rings that are positioned around the flexible elongate member. A locking area is formed by a collar or locking section 118 and knob or retention section 120 are disposed at the proximal portion 109 of the flexible elongate member 106.

The intravascular device 102 in FIG. 1 includes core wire comprising a distal core 210 and a proximal core 220. The distal core 210 and the proximal core 220 are metallic components forming part of the body of the intravascular device 102. For example, the distal core 210 and the proximal core 220 may be flexible metallic rods that provide structure for the flexible elongate member 106. The distal core 210 and/or the proximal core 220 can be made of a metal or metal alloy. For example, the distal core 210 and/or the proximal core 220 can be made of stainless steel, Nitinol, nickel-cobalt-chromium-molybdenum alloy (e.g., MP35N), and/or other suitable materials. In some embodiments, the distal core 210 and the proximal core 220 are made of the same material. In other embodiments, the distal core 210 and the proximal core 220 are made of different materials. The diameter of the distal core 210 and the proximal core 220 can vary along their respective lengths. A joint between the distal core 210 and proximal core 220 is surrounded and contained by a hypotube 215. The electronic component 112 can in some cases be positioned at a distal end of the distal core 210.

In some embodiments, the intravascular device 102 comprises a distal subassembly and a proximal subassembly that are electrically and mechanically joined together, which creates an electrical communication between the electronic component 112 and the conductive portions 132, 134. For example, flow data obtained by the electronic component 112 (in this example, electronic component 112 is a flow sensor) can be transmitted to the conductive portions 132, 134. In an exemplary embodiment, the flow sensor 112 is a single ultrasound transducer element. In some embodiments, the transducer element emits ultrasound signals, receives echoes, and generates electrical signals representative of the echoes. The processing system 306 processes the electrical signals to extract the flow velocity of the fluid. In some embodiments, the electronic component is a pressure transducer (e.g., based on piezoresistive technology) and generates electrical signals representative of the pressure within the vessel. The signal carrying filars carry these electrical signals from the sensor at the distal portion to the connector at the proximal portion.

Control signals from a processing system 306 (e.g., a processor circuit of the processing system 306) in communication with the intravascular device 102 can be transmitted to the electronic component 112 via a connector 314 that attached to the conductive portions 132, 134. The distal subassembly can include the distal core 210. The distal subassembly can also include the electronic component 112, the conductive members 230, and/or one or more layers of insulative polymer/plastic 240 surrounding the conductive members 230 and the core 210. For example, the polymer/plastic layer(s) can insulate and protect the conductive members of the multi-filar cable or conductor bundle 230. The proximal subassembly can include the proximal core 220. The proximal subassembly can also include one or more polymer layers 250 (hereinafter polymer layer 250) surrounding the proximal core 220 and/or conductive ribbons 261 embedded within the one or more insulative and/or protective polymer layer 250. In some embodiments, the proximal subassembly and the distal subassembly are separately manufactured. During the assembly process for the intravascular device 102, the proximal subassembly and the distal subassembly can be electrically and mechanically joined together. As used herein, flexible elongate member can refer to one or more components along the entire length of the intravascular device 102, one or more components of the proximal subassembly (e.g., including the proximal core 220, etc.), and/or one or more components the distal subassembly 410 (e.g., including the distal core 210, etc.). Accordingly, flexible elongate member may refer to the combined proximal and distal subassemblies described above. The joint between the proximal core 220 and distal core 210 is surrounded by the hypotube 215.

In various embodiments, the intravascular device 102 can include one, two, three, or more core wires extending along its length. For example, a single core wire can extend substantially along the entire length of the flexible elongate member 106. In such embodiments, a locking section 118 and a section 120 can be integrally formed at the proximal portion of the single core wire. The electronic component 112 can be secured at the distal portion of the single core wire. In other embodiments, such as the embodiment illustrated in FIG. 1, the locking section 118 and the section 120 can be integrally formed at the proximal portion of the proximal core 220. The electronic component 112 can be secured at the distal portion of the distal core 210. The intravascular device 102 includes one or more conductive members 230 (e.g., a multi-filar conductor bundle or cable) in communication with the electronic component 112. For example, the conductive members 230 can be one or more electrical wires that are directly in communication with the electronic component 112. In some instances, the conductive members 230 are electrically and mechanically coupled to the electronic component 112 by, e.g., soldering. In some instances, the conductor bundle 230 comprises two or three electrical wires (e.g., a bifilar cable or a trifilar cable). An individual electrical wire can include a bare metallic conductor surrounded by one or more insulating layers. The conductive members 230 can extend along the length of the distal core 210. For example, at least a portion of the conductive members 230 can be spirally wrapped around the distal core 210, minimizing or eliminating whipping of the distal core within tortuous anatomy.

The intravascular device 102 includes one or more conductive ribbons 261 at the proximal portion of the flexible elongate member 106. The conductive ribbons 261 are embedded within polymer layer 250. The conductive ribbons 261 are directly in communication with the conductive portions 132 and/or 134. In some instances, a multi-filar conductor bundle 230 is electrically and mechanically coupled to the electronic component 112 by, e.g., soldering. In some instances, the conductive portions 132 and/or 134 comprise conductive ink (e.g., metallic nano-ink, such as copper, silver, gold, or aluminum nano-ink) that is deposited or printed directed over the conductive ribbons 261.

As described herein, electrical communication between the conductive members 230 and the conductive ribbons 261 can be established at the connection portion 114 of the flexible elongate member 106. By establishing electrical communication between the conductor bundle 230 and the conductive ribbons 261, the conductive portions 132, 134 can be in electrical communication with the electronic component 112.

In some embodiments represented by FIG. 1, the intravascular device 102 includes a locking section 118 and a retention section 120. To form locking section 118, a machining process is used to remove polymer layer 250 and conductive ribbons 261 in locking section 118 and to shape proximal core 220 in locking section 118 to the desired shape. As shown in FIG. 1, locking section 118 includes a reduced diameter while retention section 120 has a diameter substantially similar to that of proximal core 220 in the connection portion 114. In some instances, because the machining process removes conductive ribbons in locking section 118, proximal ends of the conductive ribbons 261 would be exposed to moisture and/or liquids, such as blood, saline solutions, disinfectants, and/or enzyme cleaner solutions, an insulation layer 158 is formed over the proximal end portion of the connection portion 114 to insulate the exposed conductive ribbons 261.

In some embodiments, a connector 314 provides electrical connectivity between the conductive portions 132, 134 and a patient interface module (PIM) 304. The PIM 304 may in some cases connect to a console or processing system 306, which includes or is in communication with a display 308.

The system 100 may be deployed in a catheterization laboratory having a control room. The processing system 306 may be located in the control room. Optionally, the processing system 306 may be located elsewhere, such as in the catheterization laboratory itself. The catheterization laboratory may include a sterile field while its associated control room may or may not be sterile depending on the procedure to be performed and/or on the health care facility. In some embodiments, device 102 may be controlled from a remote location such as the control room, such that an operator is not required to be in close proximity to the patient.

The intraluminal device 102, PIM 304, and display 308 may be communicatively coupled directly or indirectly to the processing system 306. These elements may be communicatively coupled to the medical processing system 306 via a wired connection such as a standard copper multi-filar conductor bundle 230. The processing system 306 may be communicatively coupled to one or more data networks, e.g., a TCP/IP-based local area network (LAN). In other embodiments, different protocols may be utilized such as Synchronous Optical Networking (SONET). In some cases, the processing system 306 may be communicatively coupled to a wide area network (WAN).

The PIM 304 transfers the received signals to the processing system 306 where the information is processed and displayed (e.g., as physiology data in graphical, symbolic, or alphanumeric form) on the display 308. The console or processing system 306 can include a processor and a memory. The processing system 306 may be operable to facilitate the features of the intravascular sensing system 100 described herein. For example, the processor can execute computer readable instructions stored on the non-transitory tangible computer readable medium.

The PIM 304 facilitates communication of signals between the processing system 306 and the intraluminal device 102. The PIM 304 can be communicatively positioned between the processing system 306 and the intraluminal device 102. In some embodiments, the PIM 304 performs preliminary processing of data prior to relaying the data to the processing system 306. In examples of such embodiments, the PIM 304 performs amplification, filtering, and/or aggregating of the data. In an embodiment, the PIM 304 also supplies high-and low-voltage DC power to support operation of the intraluminal device 102 via the conductive members 230.

A multi-filar cable or transmission line bundle 230 can include a plurality of conductors, including one, two, three, four, five, six, seven, or more conductors. In the example shown in FIG. 1, the multi-filar conductor bundle 230 includes two straight portions 232 and 236, where the multi-filar conductor bundle 230 lies parallel to a longitudinal axis of the flexible elongate member 106, and a spiral portion 234, where the multi-filar conductor bundle 230 is wrapped around the exterior of the flexible elongate member 106 and then overcoated with an insulative and/or protective polymer 240. Communication, if any, along the multi-filar conductor bundle 230 may be through numerous methods or protocols, including serial, parallel, and otherwise, wherein one or more filars of the bundle 230 carry signals. One or more filars of the multi-filar conductor bundle 230 may also carry direct current (DC) power, alternating current (AC) power, or serve as a ground connection.

The display or monitor 308 may be a display device such as a computer monitor or other type of screen. The display or monitor 308 may be used to display selectable prompts, instructions, and visualizations of imaging data to a user. In some embodiments, the display 308 may be used to provide a procedure-specific workflow to a user to complete an intraluminal imaging procedure.

Before continuing, it should be noted that the examples described above are provided for purposes of illustration and are not intended to be limiting. Other devices and/or device configurations may be utilized to carry out the operations described herein.

In some aspects, the device 102 be positioned within or acquire data related to an anatomical object. The anatomical object may include any anatomy or anatomical feature, such as a kidney, liver, and/or any other anatomy of a subject. The present disclosure can be implemented in the context of any number of anatomical locations and tissue types, including without limitation, organs including the liver, kidneys, gall bladder, pancreas, lungs; ducts; intestines; nervous system structures including the brain, dural sac, spinal cord and peripheral nerves; the urinary tract; as well as valves within the blood vessels, blood, abdominal organs, and/or other systems of the body. In some aspects, the anatomical object may include malignancies such as tumors, cysts, lesions, hemorrhages, or blood pools within any part of human anatomy. The anatomy may be a blood vessel, as an artery or a vein of a subject's vascular system, including cardiac vasculature, peripheral vasculature, neural vasculature, renal vasculature, and/or any other suitable lumen inside the body. In addition to natural structures, the present disclosure can be implemented in the context of man-made structures such as, but without limitation, heart valves, stents, shunts, filters, implants and other devices.

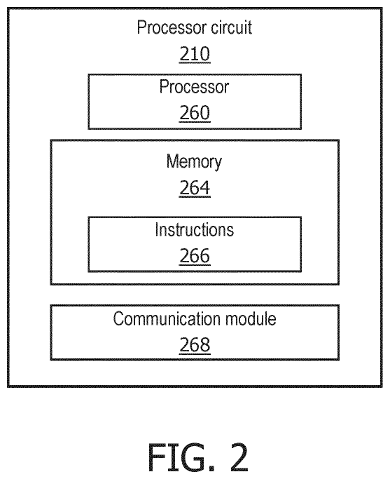

FIG. 2 is a schematic diagram of a processor circuit 210, according to aspects of the present disclosure. The processor circuit 210 may be implemented in the control system 130 of FIG. 1, the intravascular system 101 or any other suitable location. In an example, the processor circuit 210 may be in communication with intravascular device 102 and the display 308 within the system 100. One or more processor circuits 210 are configured to execute the operations described herein. As shown, the processor circuit 210 may include a processor 260, a memory 264, and a communication module 268. These elements may be in direct or indirect communication with each other, for example via one or more buses.

The processor 260 may include a CPU, a GPU, a DSP, an application-specific integrated circuit (ASIC), a controller, an FPGA, another hardware device, a firmware device, or any combination thereof configured to perform the operations described herein. The processor 260 may also be implemented as a combination of computing devices, e.g., a combination of a DSP and a microprocessor, a plurality of microprocessors, one or more microprocessors in conjunction with a DSP core, or any other such configuration.

The memory 264 may include a cache memory (e.g., a cache memory of the processor 260), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 264 includes a non-transitory computer-readable medium. The memory 264 may store instructions 266. The instructions 266 may include instructions that, when executed by the processor 260, cause the processor 260 to perform the operations described herein with reference to the probe 110 and/or the host 130 (FIG. 1). Instructions 266 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 268 can include any electronic circuitry and/or logic circuitry to facilitate direct or indirect communication of data between the processor circuit 210, the device 102, and/or the display 308. In that regard, the communication module 268 can be an input/output (I/O) device. In some instances, the communication module 268 facilitates direct or indirect communication between various elements of the processor circuit 210 and/or the device 102 (FIG. 1) and/or the connector 314, PIM 304, processing system 306, and/or display 308 (FIG. 1).

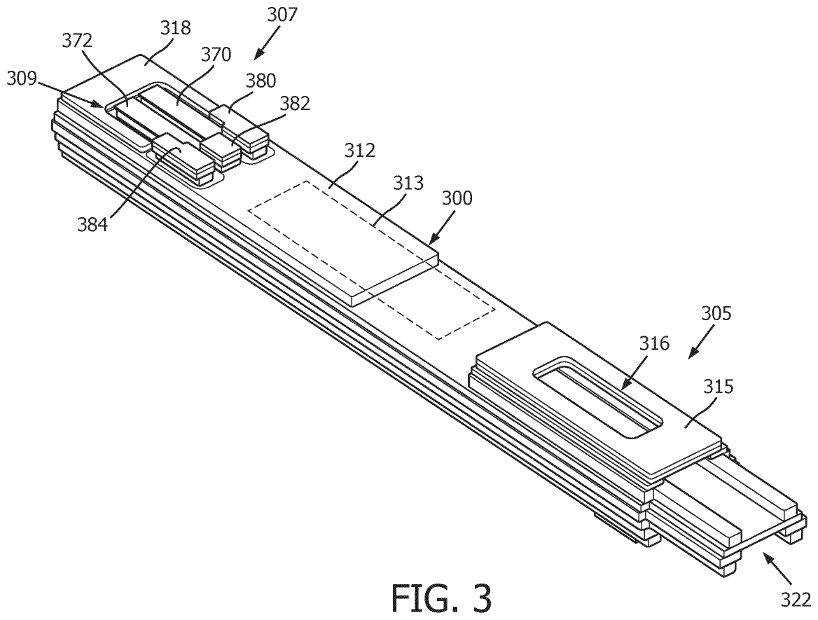

FIG. 3 is a perspective view of a sensor mount of an intravascular device, according to aspects of the present disclosure. The sensor mount 300 is designed with multiple layers that provide support and protection for a pressure sensor. For example, the sensor mount 300 can be produced using additive manufacturing, such that a layer of the sensor mount is built on another layer. Examples of sensor mounts and/or sensor housings (such as those produced by additive manufacturing, 3D printing, or semiconductor fabrication techniques) can be found in U.S. Pat. No. 10,932,678 to Burkett, U.S. Pat. No. 9,974,446 to Burkett, U.S. Provisional Patent Application No. 63/328,355, filed Apr. 7, 2022, and titled “Multi-Component Housing for Sensor in Intraluminal Device” (Attorney Docket No. 2021PF00898/44755.2271PV01), and U.S. Provisional Patent Application No. 63/330,380, filed Apr. 13, 2022, and titled “Sensor Housing for Improved Accuracy and Electrical Reliability”, each of which is hereby incorporated by reference in its entirety as though fully set forth herein. The sensor mount 300 can include a plurality of layers that are stacked on top of one another, including 2, 10, 15, 20, 50, 100, and/or other suitable values both larger and smaller. The layers of the sensor mount 300 may include any suitable materials (metallic, conductive, polymeric, non-conductive, and/or combinations thereof), depending on the application. The sensor mount 300 may serve to maintain the sensor's position, preventing movement or damage that could affect its performance.

The sensor positioned on the sensor mount 300 can obtain intraluminal (e.g., intravascular) medical data associated with the body lumen in which the intraluminal device (e.g., catheter or guidewire) is positioned. For example, pressure data obtained by a pressure sensor mounted on the sensor mount 300 can be used by a processor to calculate physiological values such as hyperemic or non-hyperemic pressure ratios (e.g., fractional flow reserve or FFR, instantaneous wave-free ratio or iFR, and/or any other distal pressure over proximal/aortic pressure or Pd/Pa). While aspects of the sensor mount 300 are described with reference to a pressure sensor, it is understood that these aspects could be described with respect to a flow sensor, temperature sensor, and/or other type of intravascular/intraluminal sensor.

The sensor mount 300 can be made from a variety of materials, including conductive and/or non-conductive materials. In some aspects, a conductive material may provide conduction/transmission of electrical signals, electrical shielding, or electrical grounding. In some aspects, a non-conductive material can provide electrical insulation. The type of material(s) used will depend on the application and the specific requirements for the sensor.

The sensor mount includes a distal region 305 terminating at a distal end and a proximal region 307 terminating at a proximal end. One or a plurality of layers a channel 322 extends from the distal region 305 and includes a layer 315 that forms an opening 316. In some aspects, the opening 316 may be configured to receive the proximal end of a shaping ribbon. The proximal end of the shaping ribbon may be coupled to the sensor mount inside of the opening 316. In some aspects, the shaping ribbon may extend distally from the sensor mount within the coil 290. The opening may be of any suitable shapes and sizes, depending on the type of sensor and the application.

The proximal region 307 includes a layer 312 and a layer 318. The layer 318 may be positioned on the upper surface of the layer 312. An opening 309 is formed within the layers 312 and 318 at the proximal region 307. Multiple conductive pads are positioned within the opening 309. The conductive pads may be referred to by any suitable term, such as bonding pads. The conductive pads may include any suitable number of conductive pads. For example, the conductive pads may include two proximal conductive pads, a conductive pad 370 and a conductive pad 372, as well as three distal conductive pads, a conductive pad 380, a conductive pad 382, and a conductive pad 384. The conductive pads 370 and 372 may be constructed of palladium. The conductive pads 380, 382, and 384 may be constructed of palladium as well, but with a thin layer or veneer of gold positioned on the upper surface of the palladium layer.

In some aspects, the height of the conductive pads 380, 382, and 384 may be greater relative to the conductive pads 370 and 372. In some aspects, the height of the conductive pads 380, 382, and 384 may be the same as the height of the layer 315. In some aspects, the height of the conductive pads 380, 382, and 384 may be the same as the height of corresponding conductive pads of a sensor assembly coupled to the sensor mount 300.

A middle region may be positioned between the distal region 305 and the proximal region 307. This middle region may include a region 313 where a pressure sensor assembly may be placed. As shown, a sensor assembly placed in the region 313 may be cantilevered such that it is mechanically coupled to the layer 312 shown. The portion of the sensor assembly which hangs over the edge of the layer 312 may include a pressure sensor. In this orientation, fluid may be allowed to surround the pressure sensor by being positioned between the lower surface of the pressure sensor and the upper surface of the sensor mount 300.

While aspects of the present disclosure are described with respect to a sensor mount for a pressure sensor that is side-facing, in some aspects, the sensor mount can be for a different type of sensor (e.g., flow sensor/ultrasound transducer for Doppler signals, temperature sensor, etc.). In some aspects, the sensor mount can be for a forward-facing sensor or a backward-facing sensor. For example, the sensor mount can be for a forward-facing flow sensor/ultrasound transducer for Doppler signals. In some aspects, an intravascular guidewire or catheter can include two sensor mounts for two different sensors (e.g., one sensor mount for a flow sensor, one sensor mount for a pressure sensor), respectively. In some aspects, an intravascular guidewire or catheter can include one sensor mount for multiple different sensors (e.g., one sensor mount for both a flow sensor and a pressure sensor).

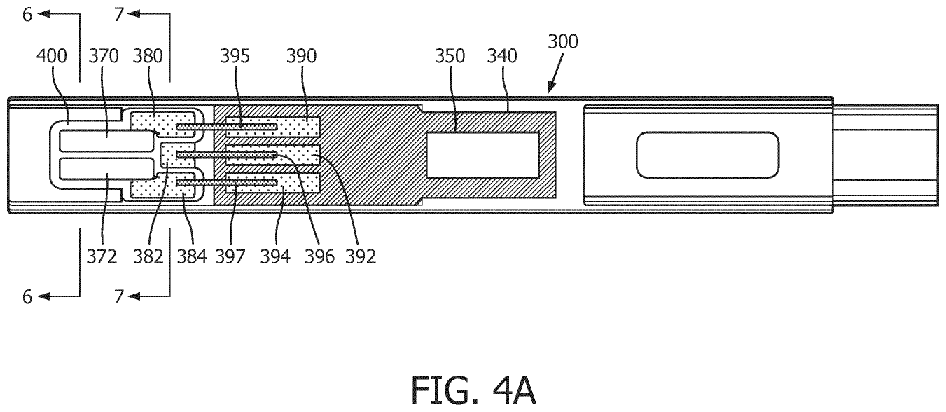

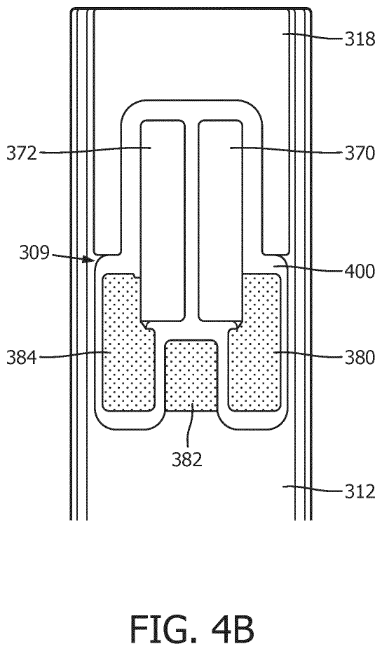

FIG. 4A is a diagrammatic top view of a sensor assembly 340 coupled to the sensor mount 300 of an intravascular device, according to aspects of the present disclosure. As shown in FIG. 4A, the sensor mount 340 is coupled to the sensor mount 300 at the location 313 (FIG. 3). The sensor assembly 340 may include a pressure sensor 350 and multiple conductive pads. In some aspects, the sensor assembly may include a number of conductive pads equal to the number of distal conductive pads of the sensor mount 300. For example, as shown in FIG. 4A, the sensor assembly may include three conductive pads including conductive pads 390, 392, and 394. In some aspects, the sensor 350 of the sensor assembly 340 may be electrically coupled to any or all of conductive pads 390, 392, and/or 394.

In some aspects, the sensor mount 300 and sensor assembly 340, in the configuration shown, may facilitate transmittal of electrical signals, including pressure measurements or data, from the sensor 350 to the connector 314, PIM 304, processing system 306, and/or display 308 (FIG. 1). For example, the conductive pads 390 and 394 may be electrically connected to positive and negative terminals of the sensor 340. The conductive pad 392 may be grounded. Wire bonds may electrically couple the sensor assembly 340 to the sensor mount 300. In particular, a conductor 395 may be bonded to the conductive pad 390 and the conductive pad 380. In this way, the conductor 395 may electrically couple the conductive pad 390 and the conductive pad 380. Similarly, a conductor 396 may be bonded to the conductive pad 392 and the conductive pad 382. In this way, the conductor 396 may electrically couple the conductive pad 392 and the conductive pad 382. A conductor 397 may be bonded to the conductive pad 394 and the conductive pad 384. In this way, the conductor 397 may electrically couple the conductive pad 394 and the conductive pad 384. The number of conductors 395, 396, 397, the number of conductive pads 390, 390, 392 (of the sensor assembly 340), and/or the number of conductive pads 380, 382, 384 (of the sensor mount 300) can be equal.

Examples of the conductors 395, 396, 397 include wire bonds or bond wires, filars, wires or wire segments, conductive traces, conductive ink, electrodes, etc. The conductors 395, 396, 397 provide signal/data communication between the conductive pads. For example, the conductive pads and the conductors 395, 396, 397 can transmit sensor data obtained by the sensor to the proximal portion of the intravascular guidewire (e.g., to the PIM 304 and/or the processing system 306). For example, the conductive pads and the conductors 395, 396, 397 can transmit control signals for the sensor to obtain sensor data from the proximal portion of the intravascular guidewire (e.g., from the PIM 304 and/or the processing system 306).

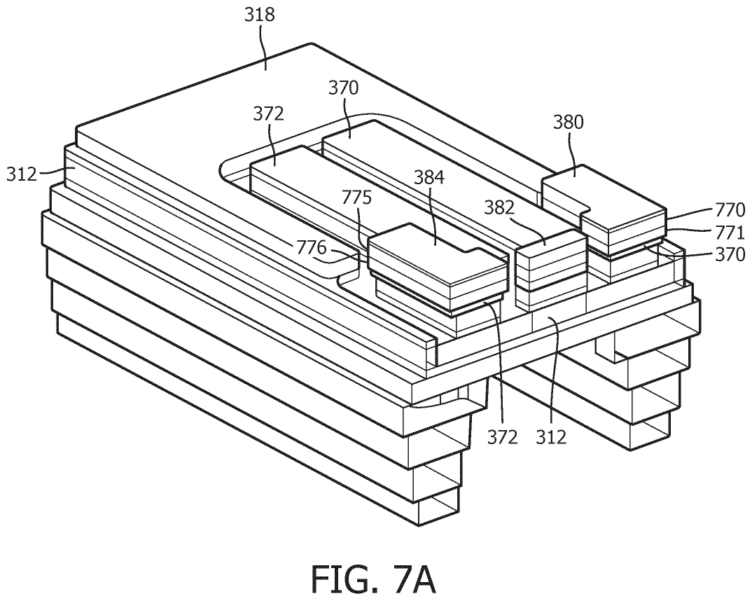

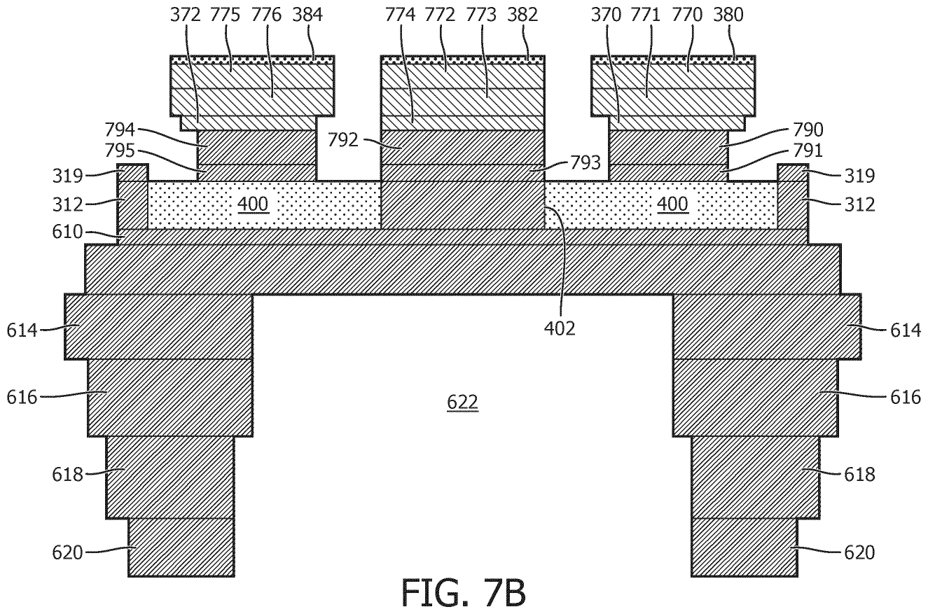

In some aspects, the conductive pad 380 may be electrically coupled to the conductive pad 370. For example, and as shown in more detail with reference to FIG. 7A, the conductive pad 370 may extend distally to form a layer of the conductive pad 380. In this way, a positive signal (or negative as the case may be) may travel from the conductive pad 380 to the conductive pad 370. Similarly, the conductive pad 384 may be electrically coupled to the conductive pad 372. For example, and as shown in more detail with reference to FIG. 7A, the conductive pad 372 may extend distally to form a layer of the conductive pad 384. In this way, a positive signal (or negative as the case may be) may travel from the conductive pad 384 to the conductive pad 372.

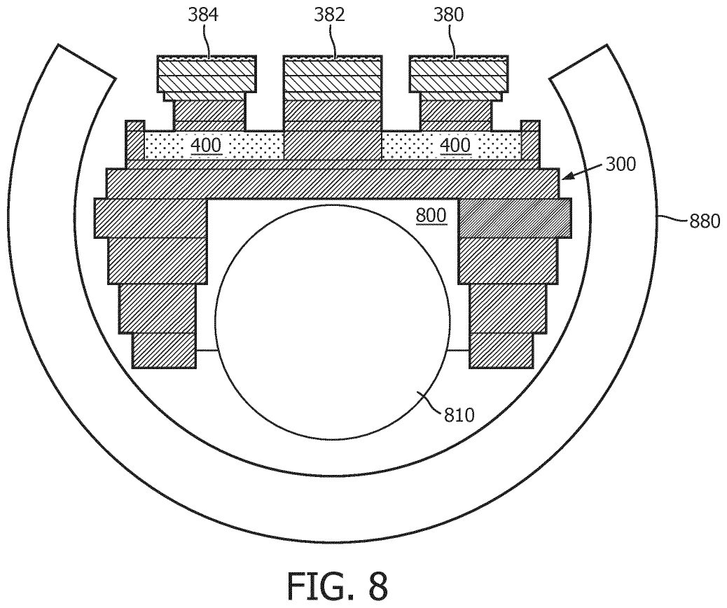

The conductive pad 382 may be electrically coupled to the sensor mount 300 and may serve as a ground. For example, as shown in more detail with reference to FIG. 7A, the conductive pad 382 may extend downward and be mechanically and electrically coupled to the sensor mount 300. As shown with reference to FIG. 8, this ground signal may be carried via solder to a distal core wire in electrical communication with a proximal core wire which is in electrical communication with a ground pad of the connector 314 (FIG. 1).

The conductive pad 370 may be electrically bonded (e.g., via solder) to a lead of a flexible conductor which runs along the flexible elongate member of the intravascular device 102 and is in electrical communication with a corresponding conductive pad of the connector 314 (FIG. 1). Similarly, the conductive pad 372 may be electrically bonded (e.g., via solder) to a lead of an additional flexible conductor which runs along the flexible elongate member of the intravascular device 102 and is in electrical communication with a corresponding conductive pad of the connector 314 (FIG. 1).

Additional aspects of the conductive pads 370, 372, 380, 382, and 384 will be described with reference to FIG. 4B.

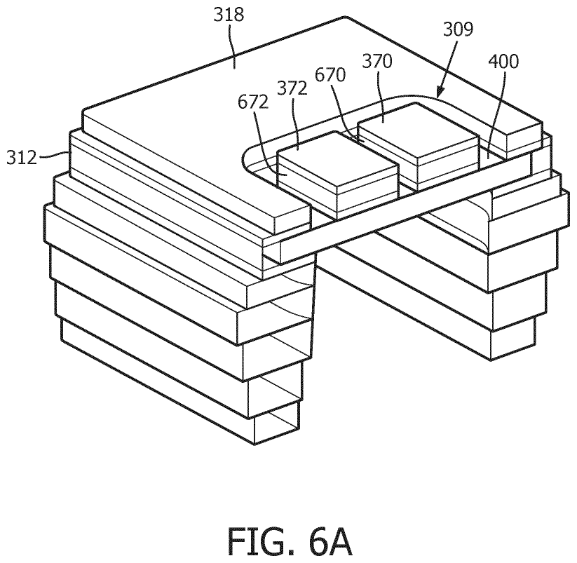

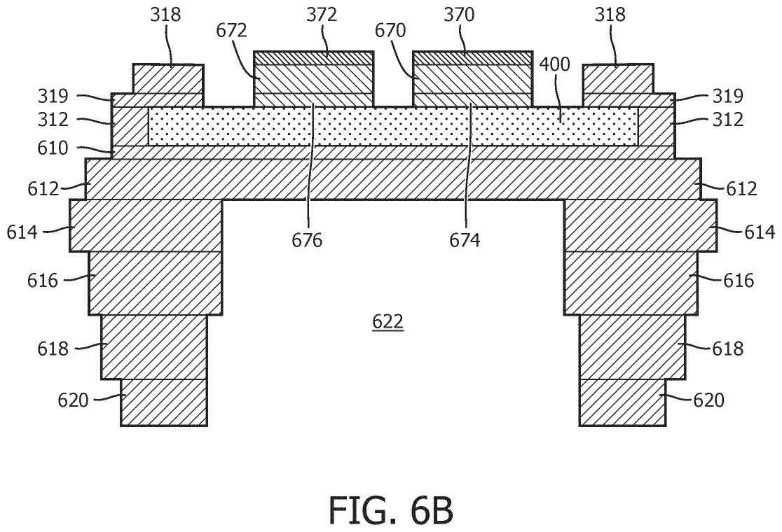

FIG. 4B is an enlarged diagrammatic top view of the sensor mount 300 of the intravascular device 102, according to aspects of the present disclosure. As shown in FIG. 4B, the layers 312 and 318 form a cavity 309 within the sensor mount 300. A dielectric filler material 400 may be positioned within the cavity 309. In some aspects, as shown and described in greater detail with reference to FIGS. 6A and 7A, the dielectric material may electrically isolate the pads 370, 372, 380, and 384 from the sensor mount 300. As explained previously, the conductive pad 382 is electrically coupled to the sensor mount 300. Therefore, the dielectric material 400 does not isolate the pad 382 from the sensor mount 300. In some aspects, the material 400 may be any suitable non-conductive material.

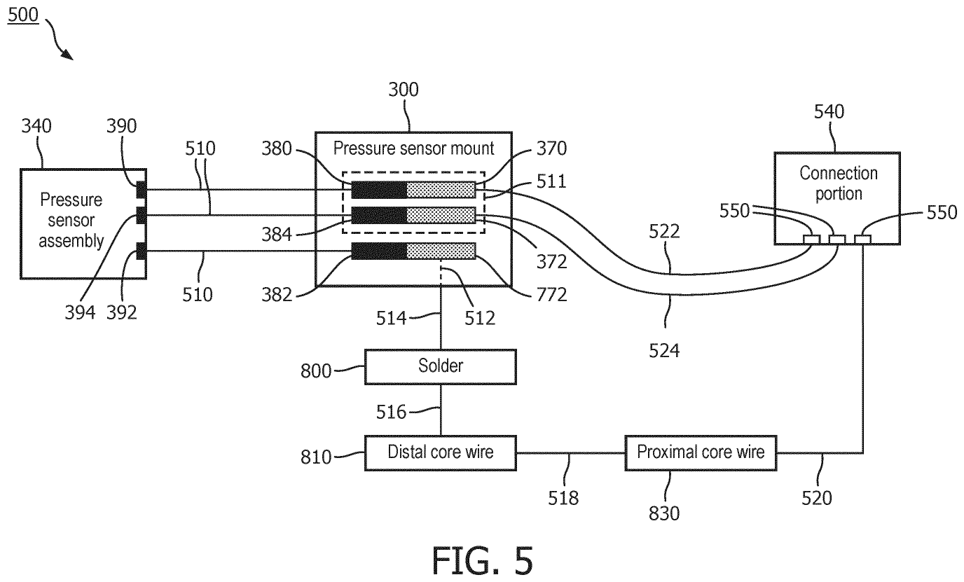

FIG. 5 is a diagrammatic view of a signal path 500 of the intravascular sensing system 100, according to aspects of the present disclosure. As shown in FIG. 5, the pressure sensor assembly 340 includes conductive pads 390, 392, and 394. The pressure sensor mount 300 includes three conductive pads 380, 384, and 382. The conductive pads 380, 384, and 382 may be gold layers adhered on a layer of palladium. For example, the conductive pad 380 may be layered over the top surface of a palladium layer which may be, or may be electrically coupled to, the conductive pad 370, the conductive pad 384 may be layered over the top surface of a palladium layer which may be, or may be electrically coupled to, the conductive pad 372, and the conductive pad 382 may be layered over the top surface of a palladium layer 772 (FIG. 7B). The conductive pads 390, 394, and 392 may be in electrical communication with the conductive pads 380, 384, and 382 via one or more wire bonds 510. In some aspects, the wire bonds 510 may be substantially similar to the wire bonds 395, 396, and 397 shown and described with reference to FIG. 4A.

In some aspects, the sensor mount 300 includes the conductive pads 370 and/or 372 (which may constitute one set of conductive pads) and the conductive pads 380, 382, and/or 384 (which may constitute another set of conductive pads). In some aspects, the sensor mount may not include the conductive pads 370 and/or 372 and only include the conductive pads 380, 382, and/or 384. In some aspects, the sensor mount may not include the conductive pads 380, 382, and/or 384 and only include the conductive pads 370 and/or 372.

In aspects in which the conductive pads 370 and/or 372 are omitted from the sensor mount, the distal ends of the conductors (e.g., conductors 230 of FIG. 1, or 522 and 524 of FIG. 5) may be directly coupled, mechanically and electrically, to the conductive pads 380 and/or 384. In some aspects, the conductive pads 380 and/or 384 may extend further proximally to provide more space for coupling of the distal ends of the conductors. In that regard, the conductive pads 380 and/or 384 may be coupled both to the distal ends of the conductors as well as the proximal ends of the wire bonds 395, 396 and/or 397 (FIG. 4A). The total number of layers stacked on top of one another could be reduced if the conductive pads 370 and/or 372 are omitted (e.g., in comparison to FIGS. 7A, 7B).

Alternatively, in aspects in which the conductive pads 380, 382, and/or 384 are omitted from the sensor mount, the total number of layers may be reduced. In such aspects, the distal ends of the conductors (e.g., conductors 230 of FIG. 1, and/or 522 and 524 of FIG. 5) may be directly coupled, mechanically and electrically, to the conductive pads 370 and/or 372. The conductive pads 370 and/or 372 may be coupled both to the distal ends of the conductors (e.g., conductors 230 of FIG. 1, and/or 522 and 524 of FIG. 5) and the proximal ends of the wire bonds 395, 396 and/or 397. In such aspects, the total number of layers may be reduced if the conductive pads 370 and/or 372 are omitted (e.g., in comparison to FIGS. 7A, 7B). For example, in some aspects, any of layers 771, 770, and 380 (FIG. 7A/7B), layers 776, 775, 384 (FIG. 7A/7B), and/or layers 774, 773, 382 (FIG. 7A/7B) may be omitted.

In some aspects, the conductive pads 380, 384, and 382 and the conductive pads 390, 394, and 392 may be constructed of gold. Similarly, the wire bonds 395, 396, and 397 may be constructed of gold. In some aspects, all of the conductive pads 380, 384, and 382 and the conductive pads 390, 394, and 392 and the wire bonds 395, 396, and 397 may be formed of a different material. In some aspects, each of the conductive pads 380, 384, and 382 and the conductive pads 390, 394, and 392 and the wire bonds 395, 396, and 397 being formed of the same material may advantageously increase the bonding strength of the wire bonds 395, 396, and 397 to the conductive pads 390, 394, and 392 and the wire bonds 395, 396, and 397. In some aspects, this may also allow the wire bonds 395, 396, and 397 to be formed between the conductive pads 390, 394, and 392 and the wire bonds 395, 396, and 397 by a machine, allowing the process to be automated rather than performed manually leading to increased reliability, decreased rate of error, and decreased manufacturing expense and time.

As shown by the box 511 in FIG. 5, the conductive pads 380, 370, 384, and 372 may be electrically isolated from the pressure sensor mount (e.g., by the dielectric material 400 described at FIG. 4B). The conductive pad 382, however, may be in electrical communication, via at least layer 772, to the sensor mount 300 itself, allowing electrical signals to pass through the sensor mount 300, as shown by the line 512.

As shown in FIG. 5, the conductive pad 370 is in electrical communication with a corresponding pad 550 of the connection portion 540 (e.g., the connector 314 of FIG. 1) via a conductor 522 extending along the flexible elongate member. The conductive pad 372 is in electrical communication with a corresponding pad 550 of the connection portion 540 (e.g., the connector 314 of FIG. 1) via a conductor 524 extending along the flexible elongate member. The pad 382, which carries a ground signal, is in electrical communication with the layer 772, which is in electrical communication with the sensor mount 300. Signals may pass from the sensor mount 300, through solder 800, and to the distal core wire 810 as shown by lines 514 and 516. The distal core wire 810 is in electrical communication with the proximal core wire 830 which is in electrical communication with a corresponding pad 550 of the connector portion 540, as shown by lines 518 and 520. In this way, the ground signal is in electrical communication with a corresponding ground pad at the connection portion 540. As a result, the number of conductors within the flexible elongate member (e.g., conductors 522 and 524) may be reduced by using the already existing distal core wire and proximal core wire to ground the pads 392 and 382.

FIG. 6A is a cutaway perspective view of the sensor mount 300, according to aspects of the present disclosure. As shown in FIG. 6A, the opening 309 contains the dielectric filler 400. In addition, the dielectric filler 400 is positioned beneath the conductive pads 370 and 372. In other words, the dielectric filler 400 is positioned between the conductive pads 370 and 372 and the sensor mount 300 itself. Because the dielectric filler 400 is nonconductive, the conductive pads 370 and 372 are electrically isolated from the sensor mount 300.

FIG. 6A also depicts layers 672 and 670 positioned beneath the pads 372 and 370 respectively. In some aspects, these layers 672 and 670 may be constructed of conductive or nonconductive material and may provide structural support for the pads 370 and 372. For example, the layers 672 and 670 may extend into the dielectric filler 400 to increase the structural strength of the pads 372 and 370 and layers 670 and 672 while still not contacting the body of the sensor mount 300 to preserve electrical isolation.

FIG. 6B is a cross-sectional side view of a sensor mount, according to aspects of the present disclosure. FIG. 6B provides a more detailed view of the different layers forming the sensor mount 300. For example, a layer 319 may be positioned between the layers 312 and 318. The layer 312 and dielectric filler 400 may be positioned over a layer 610. Layers 612, 614, 616, 618, and 620 are also depicted. In some aspects, layers 612, 614, 616, 618, and 620 may form a channel 622 extending along a longitudinal length of the sensor mount 300. As explained in more detail with reference to FIG. 8, a core wire, such as a distal core wire, may be positioned within the channel 622 during assembly.

FIG. 6B additionally depicts the pads 372 and 370 positioned over the layers 672 and 670 respectively. In some aspects, and as shown, the layers 672 and 670 may be positioned over layers 676 and 674 respectively. In some aspects, these layers 676 and 674 may extend into a portion of the filler 400 for increased structural support.

FIG. 7A is a cutaway perspective view of the sensor mount 300, according to aspects of the present disclosure. As shown in FIG. 7A, the conductive pad 370 may extend distally and form a layer of the conductive pad 380 and the conductive pad 372 may extend distally and form a layer of the conductive pad 384. For example, the conductive pad 380 may be layered over a palladium layer 770, which is layered over an additional palladium layer 771, which is positioned on a distal region of the conductive pad 370. The conductive pad 384 may similarly be layered over a palladium layer 775, which is layered over an additional palladium layer 776, which is positioned on a distal region of the conductive pad 372. In that regard, the conductive pad 370 may be in electrical communication with the conductive pad 380. The conductive pad 372 may be in electrical communication with the conductive pad 384.

FIG. 7A additionally depicts a conductive pad 382. The conductive pad 382 may be similar to the conductive pads 380 and 384 in that the conductive pad 382 may be constructed of the same or a similar material. In some aspects, the conductive pad 382 may be constructed of gold. The conductive pad 382 is positioned over a number of palladium layers, described in more detail with reference to FIG. 7B hereafter.

FIG. 7A also illustrates how the center conductive pad 382 is electrically coupled to a ground by being connected to the sensor mount 300 itself beneath the center pad 382 as will be described in more detail with reference to FIG. 7B. The sensor mount is electrically and mechanically coupled to a core wire which extends along the flexible elongate member to a ground connection, e.g., within the channel 622 shown and described with reference to FIG. 6B.

FIG. 7B is a cross-sectional side view of the sensor mount 300, according to aspects of the present disclosure. In some aspects, the cross-sectional side view shown in FIG. 7B may be similar to the cross-sectional side view shown in FIG. 6B, but at a location further distal than the location of FIG. 6B, as illustrated in FIG. 4A previously described.

FIG. 7B illustrates multiple layers associated with each of the conductive pads 384, 382, and 380. For example, the conductive pad 384 may be layered on set of layers including the layer 775, the layer 776, and a portion of the conductive pad 372 described previously. In some aspects, the layers 775 and 776, and the conductive pad 372 may be constructed of palladium. The conductive pad 372 is positioned over a layer 794 and the layer 794 is positioned over a layer 795. In some aspects, the layers 794 and 795 may be constructed of the same material as the sensor mount 300. In some aspects, the layers 794 and 795 may be conductive layers. The layer 795 is shown positioned over the dielectric filler material 400. In that regard, the layers associated with the conductive pad 384 may be electrically isolated from the sensor mount 300. In some aspects, the layer 795 may alternatively extend into the dielectric filler material 400 to increase the structural stability of the conductive layers.

Similarly, the conductive pad 380 may be layered on set of layers including the layer 770, the layer 771, and a portion of the conductive pad 370 described previously. In some aspects, the layers 770, 771, and 370 may be constructed of palladium. The conductive pad 370 is positioned over a layer 790 and the layer 790 is positioned over a layer 791. In some aspects, the layers 790 and 791 may be constructed of the same material as the sensor mount 300. In some aspects, the layers 790 and 791 may be conductive layers. The layer 791 is shown positioned over the dielectric filler material 400. In that regard, the layers associated with the conductive pad 380 may be electrically isolated from the sensor mount 300. In some aspects, the layer 791 may alternatively extend into the dielectric filler material 400 to increase the structural stability of the conductive

The conductive pad 382 is positioned over a layer 772, which is positioned over a layer 773. The layer 773 is positioned over a layer 774. Each of the layers 772, 773, and 774 may be conductive layers. For example, the layers 772, 773, and 774 may be constructed of palladium. In some aspects, the layer 774 is positioned over a layer 792 and the layer 792 is positioned over a layer 793. The layers 792 and 793 may be constructed of the same material as the sensor mount 300. In some aspects, the layer 793 is positioned over a layer 402. The layer 402 may be a layer of the sensor mount 300 and is positioned over the layer 610 previously described. In that regard, the conductive pad 382 is in electrical communication with the sensor mount 300 through the stack of layers beneath it, including the layers 772, 773, 774, 792, 793, and 402. As shown and described in more detail with reference to FIG. 8, the sensor mount 300, and by extension, the conductive pad 382 may be in electrical communication with a distal core wire extending along the flexible elongate member and connecting to a ground source.

FIG. 8 is a cross-sectional side view of the intravascular device 102, according to aspects of the present disclosure. The cross-sectional side view shown in FIG. 8 may correspond to the same location as the cross-sectional side view of FIG. 7B, however, the cross-sectional side view shown in FIG. 8 includes additional components of the device 102, including solder 800, a distal core wire 810, and a housing 880. The cross-sectional side view shown in FIG. 8 may illustrate a view of the device 102 at a stage of assembly of the device or after assembly of the device 102. As shown, during assembly, the channel 622 (FIGS. 6B, 7B) may be filled with solder 800, or any other suitable conductive material. The distal core wire 810 may then be placed within the channel 622 such that the distal core wire 810 bonds to the sensor mount 300 within the channel 622 via the solder 800. This process brings the distal core wire 810 into electrical and mechanical communication with the sensor mount 300. After the distal core wire 810 is positioned within the channel 622 and bonded to the sensor mount 300, the housing 880 may be positioned around the assembly. This may be done by sliding the housing 880 over the assembly from the distal end, or via any other suitable method. In some aspects, as shown in FIG. 5 and described previously, the distal core wire 810 may be electrically and mechanically coupled to a proximal core wire which is connected to a connection portion of the system.

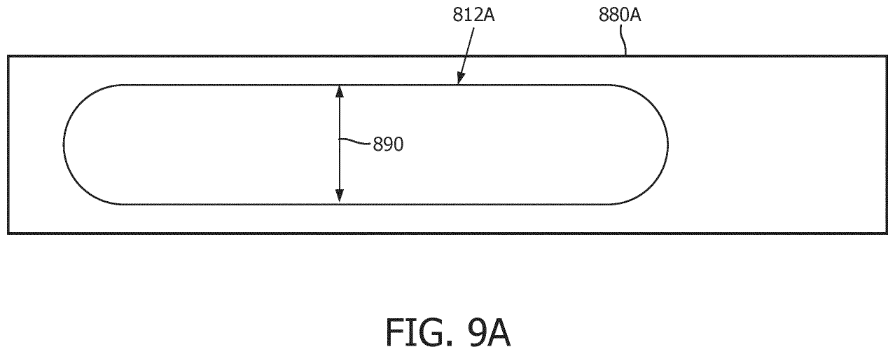

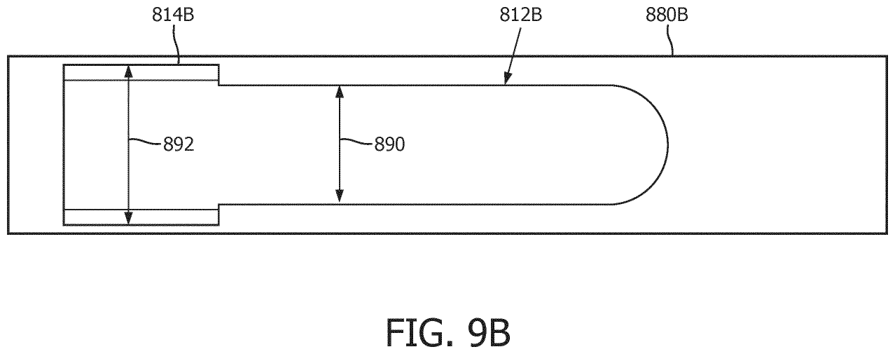

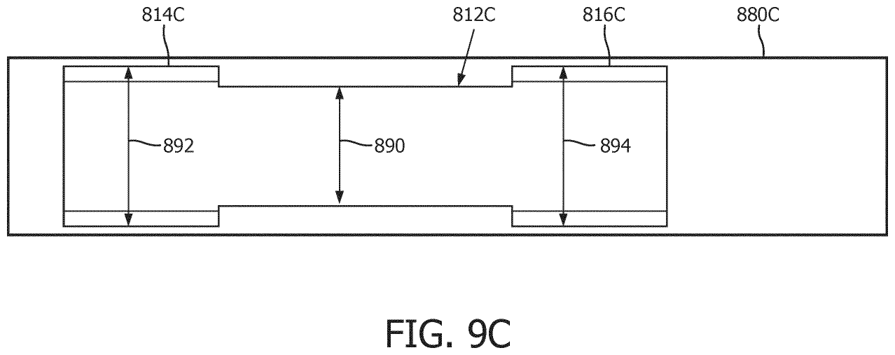

FIG. 9A is a top view of a housing 880A of the intravascular device 102, according to aspects of the present disclosure. As shown, the housing 880A may be a cylindrical part including a central lumen into which the assembly of the sensor mount 300 and core wire 810 (FIG. 8) may be positioned. The housing 880A may include an opening 812A. The opening 812A may be sized and shaped such that the various components mounted on the sensor mount 300 are exposed to the surrounding environment. For example, the opening 812A may be sized and shaped such that the sensor 350, conductive pads 390, 392, and 394, conductive pads 380, 382, and 384, and conductive pads 370 and 372 (FIG. 4A) are exposed to the surrounding environment. This opening 812A may thus allow fluid from the environment, such as blood, to come into contact with the sensor 350 and/or any suitable film positioned around the sensor 350, so the sensor 350 may acquire pressure data of the fluid. The exposed conductive pads may allow additional assembly to take place after the housing 880A has been installed. For example, wire bonds 395, 396, and/or 397 may be created by a machine or manually after the housing 880A is positioned around the assembly. In that regard, the width 890 may be of a suitable width to allow each of these components to exposed.

FIG. 9B is a top view of a housing 880B of the intravascular device 102, according to aspects of the present disclosure. In some aspects, the housing 880B may be similar to the housing 880A described previously. For example, the housing 880B may be a cylindrical part including a central lumen into which the assembly of the sensor mount 300 and core wire 810 (FIG. 8) may be positioned. The housing 880B may also include an opening 812B. However, the shape of the opening 812B may differ from the opening 812A described previously. In particular, the opening 812B may include a proximal region 814B of a width 892. The width 892 may be greater than the width 890, as shown in FIG. 9B. The increased width 892 of the region 814B may provided increased space for tool access during assembly. For example, the increased space provided by the increased width 892 may advantageously allow automated machinery to access the region of the sensor mount 300 near the proximal region 814B to install or assembly various components. For example, an automated machine may create the wire bonds 395, 396, and/or 397 (FIG. 4A) by positioning appropriate tools through the region 814B. The additional space provided by the wider opening 814B provides more space for the wire bonding process. In some aspects, the size of the wire-bonding tip of an automated machine may be larger than the width 890. In that regard, the larger section of 814B may accommodate for the tip.

FIG. 9C is a top view of a housing 880C of the intravascular device 102, according to aspects of the present disclosure. In some aspects, the housing 880C may be similar to the housings 880A and/or 880B described previously. For example, the housing 880C may be a cylindrical part including a central lumen into which the assembly of the sensor mount 300 and core wire 810 (FIG. 8) may be positioned. The housing 880C may include an opening 812C substantially similar to the proximal opening 814B described with reference to FIG. 9B. For example, the region 814C may be of a width 892. However, the housing 880C may additionally include a distal region 816C. The region 816C may be of a width 894. In some aspects, the width 894 may be substantially similar to the width 892. In some aspects, the width 894 may be the same as the width 892. In some aspects, the width 894 may be larger or smaller than the width 892.

The increased width of the distal region 816C may advantageously allow air trapped within the housing 880C during assembly of the device 102 to escape. By allowing air to escape from the housing 880C, the structural integrity of the device 102 may be increased. This makes the device 102 more reliable and able to withstand the various forces applied to the device 102 during an intravascular measurement procedure. In some aspects, the length of the opening 812C may be of the same width. In some aspects, the width 890 may be selected to be less than the widths 892 and 894 to provide additional protection to the pressure sensor (e.g., sensor 350 of FIG. 4A).

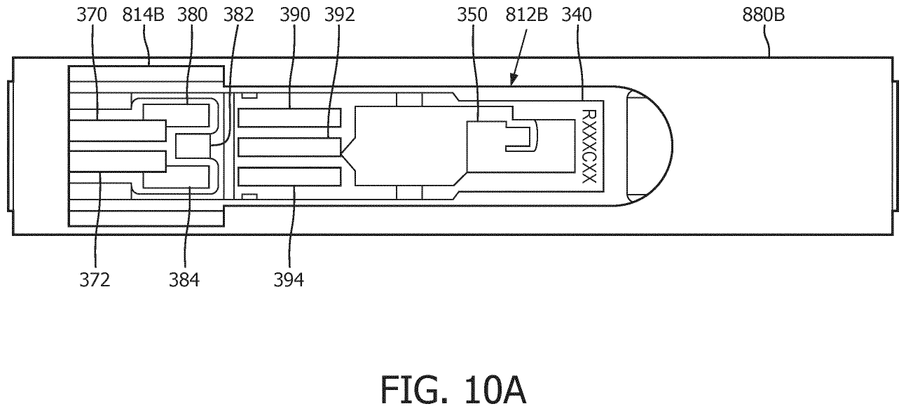

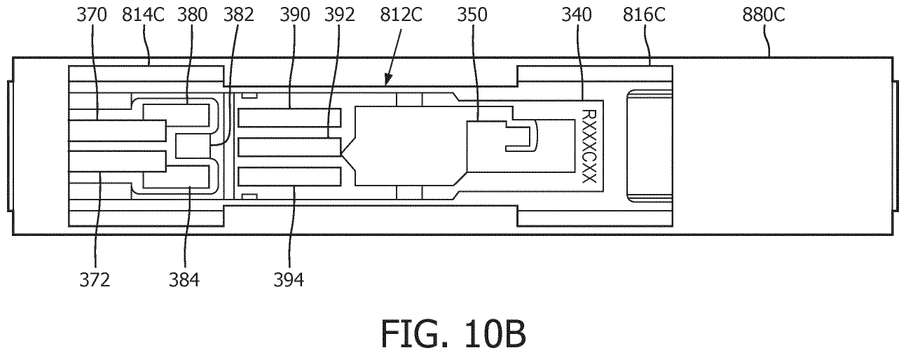

FIG. 10A is a top view of a sensor assembly 340 and housing 880B of the intravascular device 102, according to aspects of the present disclosure. For example, the view shown in FIG. 10A may be of the device 102 after complete assembly of the device 102. For example, the sensor mount 300 is positioned within the housing 880B. As shown, the opening 812B may be aligned with the sensor mount such that the sensor 340 of the sensor assembly 340 and conductive pads 390, 392, 394, 380, 382, 384, 370, and 372 are exposed. In addition, the sensor mount 300 may be positioned relative to the housing 880B such that the conductive pads are positioned proximate to the proximal region 814B of increased width allowing increased access for machine tools to perform additional steps of assembly.

FIG. 10B is a top view of a sensor assembly 350 and housing 880C of the intravascular device 102, according to aspects of the present disclosure. For example, the view shown in FIG. 10B may be of the device 102 after complete assembly of the device 102. For example, the sensor mount 300 is positioned within the housing 880C. As shown, the opening 812C may be aligned with the sensor mount such that the sensor 350 of the sensor assembly 340 and conductive pads 390, 392, 394, 380, 382, 384, 370, and 372 are exposed. In addition, the sensor mount 300 may be positioned relative to the housing 880C such that the conductive pads are positioned proximate to the region 814C of increased width allowing increased access for machine tools to perform additional steps of assembly. The distal region 816C may be positioned near a distal region of the sensor assembly 340 so as to facilitate the escape of trapped air at or near that region.

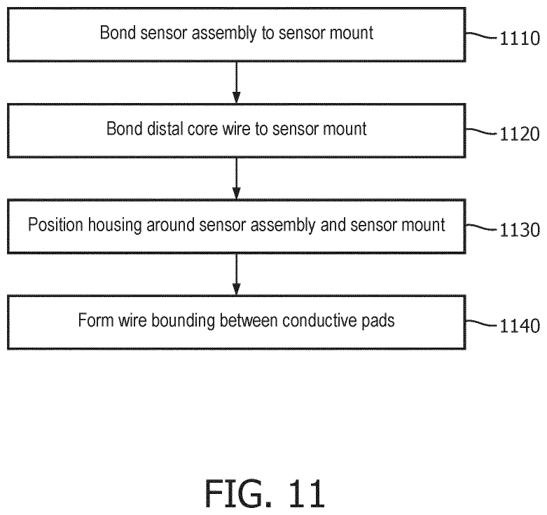

FIG. 11 is a flow diagram of a method 1100 of assembling an intravascular device 102, according to aspects of the present disclosure. As illustrated, the method 1100 includes a number of enumerated steps, but aspects of the method 1100 may include additional steps before, after, or in between the enumerated steps. In some aspects, one or more of the enumerated steps may be omitted, performed in a different order, or performed concurrently. The steps of the method 1100 can be carried out by any suitable component within the system 100 and all steps need not be carried out by the same component. In some aspects, one or more steps of the method 1100 can be performed by, or at the direction of, a processor circuit, including, e.g., the processor 210.

At step 1110, the method 1100 includes bonding a sensor assembly to a sensor mount. In some aspects, the step 1110 may be performed by an automated machine. In some aspects, the step 1110 may be performed via a pick and place process. Step 1110 may be performed by an automated machine. The process of bonding the sensor assembly to the sensor mount can be done using equipment that is specifically designed for this purpose. Such equipment might include machines that use adhesive dispensers, heat sources, or other specialized tools to bond the sensor assembly to the mount. In other cases, the step 1110 may be performed via a pick and place process. This means that the sensor assembly is moved and positioned using a machine that can pick up the assembly and place it precisely where it needs to go on the sensor mount.

At step 1120, the method 1100 includes bonding a distal core wire to the sensor mount. The distal core wire may be bonded to the sensor mount in any suitable way. For example, the distal core wire may be bonded to the sensor mount via solder. The distal core wire serves to transmit signals, such as a ground signal, from the sensor element to other components or processing units in the device or system. Multiple techniques or methods may be used to attach the wire to the mount, depending on the specific requirements of the device or system being built. Some examples of bonding methods might include soldering, welding, or adhesive bonding. The distal core wire may be securely attached to the mount to ensure reliable signal transmission.

At step 1130, the method 1100 includes positioning a housing around the sensor assembly and sensor mount. The housing may include any suitable type of housing, such as the housings 880 described herein. The housing may be positioned around the sensor assembly and sensor mount in any suitable way. For example, the housing may be a snap-on housing designed to fit snugly around the sensor mount and assembly, and it is held in place by snapping onto the mount. In some aspects, the housing may be positioned via a fastener, such as a screw, an adhesive, or any other process, such as over molding.

At step 1140, the method 1100 includes forming wire bonding between conductive pads. For example, step 1140 may include forming the wire bond 395 between the conductive pad 390 of the sensor assembly 340, forming the wire bond 396 between the conductive pad 392 and the conductive pad 382, and forming the wire bond 397 between the conductive pad 394 and the conductive pad 384.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal device, comprising:
a flexible elongate member configured to be positioned within a body lumen of a patient;
a sensor mount positioned at a distal portion of the flexible elongate member, wherein the sensor mount comprises a first plurality of conductive pads; and
a sensor assembly positioned on the sensor mount, wherein the sensor assembly comprises:
a sensor configured to obtain intraluminal medical data associated with the body lumen; and
a second plurality of conductive pads electrically coupled to the first plurality of conductive pads.

2. The intraluminal device of claim 1, wherein a number of the second plurality of conductive pads is equal to a number of the first plurality of conductive pads.

3. The intraluminal device of claim 1, wherein the sensor mount further comprises a third plurality of conductive pads.

4. The intraluminal device of claim 3, wherein the third plurality of conductive pads includes a first conductive pad, a second conductive pad, and a third conductive pad.

5. The intraluminal device of claim 4, wherein a portion of one of the conductive pads of the first plurality of conductive pads forms a layer of the first conductive pad of the third plurality of conductive pads.

6. The intraluminal device of claim 3, wherein a portion of one of the conductive pads of the first plurality of conductive pads forms a layer of the third conductive pad of the third plurality of conductive pads.

7. The intraluminal device of claim 3, wherein the second conductive pad of the third plurality of conductive pads is electrically coupled to ground.

8. The intraluminal device of claim 1, wherein the first plurality of conductive pads are positioned within a cavity of the sensor mount.

9. The intraluminal device of claim 8, wherein a dielectric filler is positioned within the cavity of the sensor mount.

10. The intraluminal device of claim 9, wherein the dielectric filler electrically insulates the first plurality of conductive pads from the sensor mount.

11. The intraluminal device of claim 1, wherein the first plurality of conductive pads includes three conductive pads.

12. The intraluminal device of claim 11, wherein two of the first plurality of conductive pads are electrically coupled to a corresponding cable extending along the flexible elongate member.

13. The intraluminal device of claim 1, wherein the sensor assembly is configured to be mechanically coupled to the sensor mount via an adhesive.

14. The intravascular device of claim 1, wherein the sensor mount is positioned within a housing.

15. The intravascular device of claim 1, wherein the housing includes an opening with a proximal and distal end, the proximal end positioned adjacent to the first plurality of conductive pads of the sensor mount.

16. The intravascular device of claim 15, wherein a width of the proximal end of the opening is wider than greater than a width of a center portion of the opening.

17. An apparatus, comprising:
an intravascular guidewire configured to be positioned within a blood vessel and comprising:
a sensor mount comprising:
a first plurality of conductive pads; and
a second plurality of conductive pads positioned distal to the first plurality of conductive pads; and
an intravascular pressure sensor disposed on the sensor mount and comprising a third plurality of conductive pads electrically coupled to the second plurality of conductive pads,
wherein the first plurality of conductive pads is electrically coupled to a corresponding plurality of conductors extending along a length of the intravascular guidewire.

18. The apparatus of claim 17, wherein a number of the second plurality of conductive pads is greater than a number of the first plurality of conductive pads.
